(19) United States
(12) Patent Application Publication
(10) Pub. No.: US 2018/0055754 A1
(43) Pub. Date: Mar. 1, 2018

(54) AQUEOUS NAIL COMPOSITIONS CONTAINING ORGANIC COLORING AGENT

(71) Applicant: L'ORÉAL, Paris (FR)

(72) Inventors: Christopher Michael MACNEILL, Fanwood, NJ (US); XianZhi ZHOU, Millburn, NJ (US); Chunhua LI, Hillsborough, NJ (US); Hy Si BUI, Piscataway, NJ (US)

(73) Assignee: L'ORÉAL, Paris (FR)

(21) Appl. No.: 15/249,886

(22) Filed: Aug. 29, 2016

(51) Int. Cl. A61K 8/81; A61K 8/46; A61K 8/37; A61Q 3/02

(52) U.S. Cl. CPC A61K 8/8141; A61K 8/466; A61K 8/37; A61K 2800/54; A61K 2800/43; A61K 2800/884; A61Q 3/02

(57) ABSTRACT

The invention relates to nail compositions including water, at least one water-soluble organic coloring agent and at least one film forming agent containing styrene, wherein the film forming agent minimizes staining of a nail by the water-soluble organic coloring agent to which the composition has been applied.

### FIELD OF THE INVENTION

The present invention relates to nail compositions comprising water, at least one water-soluble organic coloring agent and at least one film forming agent comprising styrene, wherein the film forming agent minimizes staining of a nail by the water-soluble organic coloring agent to which the composition has been applied.

### DISCUSSION OF THE BACKGROUND

Organic lakes and other organic pigments contain dyes that are functionalized with carboxylic acid and/or sulfonic acid groups, rendering them soluble in aqueous compositions. These coloring agents have great potential to stain keratin materials (for example, nails) to which they have been applied owing to the leaching of dye into the aqueous portion of the composition, and to the dye's penetrating the surface of the nails.

In the past, attempts to minimize such staining of nails have included:

Using a primer or basecoat to block penetration of the dye into the nail. For example, WO 2014/088568 relates to a nail composition set that contains at least one color coat comprising latex and water, and at least one primer/basecoat containing polyurethane;

Adhering the dye to an inorganic substrate, such as a metal oxide, through ionic, van der waals, pi-pi or other interactions;

Treating pigment with a polymer to decrease leaching of the dye from the pigment. For example, high and low density polyethylene matrices have been utilized to encapsulate Blue 1 dyes in an attempt to prevent leaching in compositions such as toothpaste (see, U.S. Pat. No. 5,876,701, U.S. Pat. No. 6,037,000, and WO 1998/051270); and

Using pH as a way to minimizing staining (see, WO 2002/089752).

Despite these attempts, there remains a need for nail compositions containing water and water-soluble organic coloring agents which result in minimal (if any) staining on nails to which they have been applied.

### SUMMARY OF THE INVENTION

The present invention relates to a nail composition comprising water, at least one water-soluble organic coloring agent and at least one film forming agent comprising styrene. The nail composition preferably further comprises at least one plasticizer and/or coalescent agent.

The present invention also relates to a nail composition set comprising (1) at least one nail composition comprising water, at least one water-soluble organic coloring agent and at least one film forming agent comprising styrene, and (2) at least one topcoat. The nail composition preferably further comprises at least one plasticizer and/or coalescent agent.

The present invention also relates to a kit comprising (1) at least one nail composition comprising water, at least one water-soluble organic coloring agent and at least one film forming agent comprising styrene, and (2) at least one topcoat. The nail composition preferably further comprises at least one plasticizer and/or coalescent agent.

The present invention further relates to methods for making up and/or protecting nails comprising applying to the nails at least one nail composition comprising water, at least one water-soluble organic coloring agent and at least one film forming agent comprising styrene. The nail composition preferably further comprises at least one plasticizer and/or coalescent agent.

The present invention also relates to methods of minimizing staining of nails by a nail composition comprising water and at least one water-soluble organic coloring agent comprising adding at least one film forming agent comprising styrene to the nail composition in an amount sufficient to minimize staining of the nail.

The present invention also relates to methods of making a nail composition comprising combining ingredients comprising water, at least one water-soluble organic coloring agent, and at least one film forming agent comprising styrene to form a nail composition.

It is to be understood that both the foregoing general description and the following detailed description are exemplary and explanatory only, and are not restrictive of the invention.

### DETAILED DESCRIPTION OF THE INVENTION

As used herein, the expression “at least one” means one or more and thus includes individual components as well as mixtures/combinations.

Other than in the operating examples, or where otherwise indicated, all numbers expressing quantities of ingredients and/or reaction conditions are to be understood as being modified in all instances by the term “about,” meaning within 10% to 15% of the indicated number.

“Film former” or “film forming agent” as used herein means a polymer or resin that leaves a film on the substrate to which it is applied, for example, after a solvent accompanying the film former has evaporated, absorbed into and/or dissipated on the substrate.

“Makeup Result” as used herein, refers to compositions where color remains the same or substantially the same as at the time of application, as viewed by the naked eye, after an extended period of time. “Makeup Result” may be evaluated by evaluating long wear properties by any method known in the art for evaluating such properties. For example, long wear may be evaluated by a test involving the application of a composition to nails and evaluating the color of the composition after an extended period of time. For example, the color of a composition may be evaluated immediately following application to nails and these characteristics may then be re-evaluated and compared after a certain amount of time. Further, these characteristics may be evaluated with respect to other compositions, such as commercially available compositions.

“Adhesion” as used herein, refers to chemical or physical bonding between a coating and a substrate. Good adhesion between nail polish and nail surface should translate to good wear properties on consumers. Adhesion properties can be quantified by in-vitro method such as a cross-cut adhesion test. In the test, a lattice pattern is cut into the coating and penetrates through to the substrate. A pressure sensitive tape is applied to the sample and then pulled off. The adhesion property can be quantified by the area of the coating remaining after peeling. For example, if the whole film remains after peeling, it indicates excellent adhesion. If most of the film gets peeled off, it indicates poor adhesion. The cross-cut test is an industrial standard test for testing adhesion for coatings. (Reference # ISO/DIN 2409, ASTM D3359).

“Substituted” as used herein, means comprising at least one substituent. Non-limiting examples of substituents for substitution include atoms, such as oxygen atoms and nitrogen atoms, as well as functional groups, such as hydroxyl groups, ether groups, alkoxy groups, acyloxyalky groups, oxyalkylene groups, polyoxyalkylene groups, carboxylic acid groups, amine groups, acylamino groups, amide groups, halogen containing groups, ester groups, thiol groups, sulphonate groups, thiosulphate groups, siloxane groups, and polysiloxane groups. The substituent(s) may be further substituted.

“Volatile”, as used herein, means having a flash point of less than about 100° C.

“Non-volatile”, as used herein, means having a flash point of greater than about 100° C.

“Nail” as used herein includes fingernails as well as toenails.

The compositions and methods of the present invention can comprise, consist of, or consist essentially of the essential elements and limitations of the invention described herein, as well as any additional or optional ingredients, components, or limitations described herein or otherwise useful.

Nail Composition

According to the present invention, a nail composition for application to nails is provided. In accordance with the present invention, the nail composition comprises (1) water, (2) at least one water-soluble organic coloring agent; and (3) at least one film forming agent comprising styrene. The nail composition preferably further comprises (4) at least one plasticizer and/or (5) at least one coalescent agent. According to preferred embodiments of the present invention, the nail compositions have a basic pH, preferably 7.5 or greater, preferably 8.0 or greater, and preferably 8.5 or greater.

Water

According to the present invention, nail compositions comprising water are provided. Preferably, total water content present in the nail compositions is in an amount ranging from about 10% to about 90%, more preferably from about 25% to about 70%, and more preferably from about 30% to about 65%, by weight, based on the total weight of the composition, including all ranges and subranges in between.

Water-Soluble Organic Coloring Agent

According to the present invention, nail compositions comprising at least one water-soluble organic coloring agent are provided. Preferably, the water-soluble organic coloring agent comprises at least one aromatic group such as a phenyl or benzyl group. Suitable examples of water-soluble organic coloring agents include, but are not limited to, blue coloring agents such as Blue 1, red coloring agents such as Red 6, and yellow coloring agents such as Yellow 5.

According to preferred embodiments, the at least one water-soluble organic coloring agent is not encapsulated.

According to preferred embodiments of the present invention, the water-soluble organic coloring agent(s) are present in the nail compositions of the present invention in an amount ranging from about 0.5% to about 10%, more preferably from about 0.75% to about 7.5%, and more preferably from about 1% to about 5%, by weight, based on the total weight of the composition, including all ranges and subranges in between.

According to preferred embodiments, prior to adding the at least one water-soluble coloring agent during preparation of a nail composition of the present invention, the water-soluble coloring agent(s) are prepared in the form of a colorant paste to help disperse the coloring agent(s) within the nail composition upon addition. For example, water-soluble organic coloring agent(s) such as Sensient Blue 1 Lake powder, Sun Chemical Blue 1 Lake powder or Kobo Blue 1 Lake powder can be added to a mixture containing high viscosity film forming agent and other ingredients such as caprylol glycol and phenoxyethanol under stirring so that that water-soluble organic coloring is well-suspended and/or the colorant paste has no visible lumps prior to adding the paste during preparation of the nail composition.

Film-Forming Agent Comprising Styrene

According to the present invention, nail compositions comprising at least one film forming agent comprising styrene are provided. Suitable examples include, but are not limited to, styrene/acrylic/ammonium methacrylic copolymers, styrene/acrylic copolymers, modified styrene/acrylic/ammonium methacrylic copolymers, modified styrene/acrylic copolymers, and mixtures thereof. It should be understood that, as used above, “(meth)acrylic” refers both to (meth)acrylic and (meth)acrylate.

According to preferred embodiments, the at least one film forming agent comprising styrene contains more than 50% styrene by weight, preferably 60% or more styrene by weight, preferably 70% or more styrene by weight, and preferably 80% or more styrene by weight, with respect to the total weight of the film forming agent. Representative examples of specific film forming agents comprising styrene include, but are not limited to, acrylic copolymer dispersions sold under the names Neocryl XK-90® (INCI name: acrylic/styrene copolymer), Neocryl A-1070® (INCI name: acrylic/styrene copolymer), Neocryl A-1090® (INCI name: acrylic/styrene copolymer), Neocryl BT-62® (INCI name: acrylic/styrene copolymer), Neocryl A-1079® (INCI name: acrylic/styrene copolymer), Neocryl A-523® (INCI name: acrylic/styrene copolymer) by the company Avecia-Neoresins, Dow Latex 432® (INCI name: Styrene/Acrylates Copolymer) by the company Dow Chemical, Neocryl A-2091 (INCI name: Modified Styrene Acrylic Copolymer Dispersion) SYNTRAN™ 5760 (styrene/acrylates/ammonium methacrylate copolymer (and) butylene glycol (and) sodium laureth-12 sulphate (INCI name), commercially available from Interpolymer Corporation); JONCRYL™ 77 (styrene/acrylates copolymer in the form of an ammonia salt, along with water and polypropylene glycol, available from BASF Performance Chemicals); and RHOPLEX™ P376 (styrene/acrylates copolymer available from Dow Chemical Company).

According to preferred embodiments, the at least one film forming agents comprising styrene is present in the nail compositions of the present invention in an active amount ranging from about 10% to about 50% by weight, preferably from about 20% to about 45% by weight, and preferably from about 30% to about 40% by weight relative to the total weight of the composition, including all ranges and subranges therebetween.

According to preferred embodiments, more film forming agent comprising styrene is present in the nail compositions of the present invention than water-soluble organic coloring agent. Preferably, the film forming agent comprising styrene and water-soluble organic coloring agent are present in a weight ratio of about 50:1 to about 5:1, preferably about 35:1 to about 7:1, and preferably about 25:1 to about 10:1, including all ranges and subranges therebetween. It is to be understood that the above weight ratios are based on the amount of active ingredient present in the compositions, not the total amount of commercial product containing such ingredients as well as additional ingredients.

Plasticizers and/or Coalescent Agents

According to particularly preferred embodiments of the present invention, the nail composition further comprises one or more ingredients selected from the group consisting of coalescent agents, plasticizers, and mixtures thereof.

Plasticizers are additives used to optimize the mechanical properties of the films. They tend to reduce the Glass Transition Temperature (Tg) and increase the softness and flexibility of the films. Coalescents are additives used to aid the coalescence of the latex particles, and hence assisting the film formation process.

Preferably, the plasticizer has a distribution coefficient D of less than or equal to 0.1. The distribution coefficient can be determined in accordance with the teaching of “A method to predict the distribution coefficient of coalescing agents between latex particles and the water phase,” Progress in Organic Coatings, vol. 30, 1997, pp. 173-177, the disclosure of which is specifically incorporated by reference herein.

Preferably, the plasticizer has a boiling point measured at ambient pressure of less than or equal to 285° C., preferably less than or equal to 270° C., and preferably less than or equal to 250° C. In the present specification, the boiling point values are to be considered accurate to ±2° C. owing to the uncertainties of boiling point measurement.

Any plasticizing agent typically found in nail polish compositions can be used. Examples of suitable plasticizers include, but are not limited to, glycols and their ester derivatives, esters of acids, in particular carboxylic acids, such as citrates, adipates, carbonates, tartrates, phosphates or sebacates, oxyethylenated derivatives, such as oxyethylenated oils, and their mixtures. For example, suitable plasticizing agents include, but are not limited to, diisobutyl adipate, the ester of teributyl acid and 2,2,4-trimethylpentane-1,3-diol, diethyl adipate, diethyl phthalate, dibutyl phthalate, dioctyl phthalate, butyl 2-ethylhexyl phthalate, dimethyl sebacate, dibutyl sebacate, ethyl stearate, 2-ethylhexyl palmitate, dipropylene glycol n-butyl ether, tributyl phosphate, tributoxyethyl phosphate, tricresyl phosphate, triphenyl phosphate, glycerol triacetate, butyl stearate, butyl glycolate, benzyl benzoate, butyl acetyltricinoleate, glyceryl acetyltricinoleate, dibutyl phthalate, diisobutyl phthalate, dioctyl phthalate, dimethoxyethyl phthalate, diamyl phthalate, triethyl citrate, tributyl citrate, tributyl acetylcitrate, tri(2-ethylhexyl) acetylcitrate, dibutyl tartrate, camphor, and mixtures thereof.

In accordance with preferred embodiments, the plasticizer, if present, is preferably present in the nail composition in an amount not greater than 10% by weight, preferably not greater than 7.5% by weight, and preferably not greater than 5% by weight with respect to the entire weight of the composition such as, for example, from 0.1% to 10% by weight, preferably from 0.25% to 7.5% by weight, and preferably from 0.5 to 5% by weight, of the total weight of the composition, including all ranges and subranges therebetween.

According to particularly preferred embodiments of the present application, nail compositions further comprising at least one coalescent agent are provided.

Preferably, the coalescent agent has a distribution coefficient D′ of greater than or equal to 0.5, measured in accordance with the above-referenced “A method to predict the distribution coefficient of coalescing agents between latex particles and the water phase,” Progress in Organic Coatings, vol. 30, 1997, pp. 173-177.

Preferably, the coalescent agent has a boiling point measured at ambient pressure ranging from 90° C. to 180° C., preferably from 150° C. to 180° C.

Any coalescent agent typically found in nail polish compositions can be used. Examples of suitable plasticizers include, but are not limited to, propylene glycol n-butyl ether, dipropylene glycol dimethyl ether, propylene glycol methyl ether acetate, propylene glycol propyl ether, methyl lactate, ethyl lactate, isopropyl lactate, and mixtures thereof.

In accordance with preferred embodiments, the coalescent agent, if present, is preferably present in the primer composition in an amount of from 0.1% to 20% by weight, preferably from 0.5% to 15% by weight, preferably from 1 to 10% by weight, of the total weight of the composition, including all ranges and subranges therebetween.

According to preferred embodiments, more film forming agent comprising styrene is present in the nail compositions of the present invention than coalescent agent. Preferably, the film forming agent comprising styrene and coalescent agent are present in an weight ratio of about 50:1 to about 5:1, preferably about 35:1 to about 7:1, and preferably about 25:1 to about 10:1. It is to be understood that the above weight ratios are based on the amount of active ingredient present in the compositions, not the total amount of commercial product containing such ingredients as well as additional ingredients.

Auxiliaries/Additives

The nail compositions of the present invention may additionally comprise an additive or auxiliary commonly used in cosmetic compositions and known to a person skilled in the art as being capable of being incorporated into a nail polish or varnish composition. Such additives or auxiliaries may be chosen from inorganic colorants, water-soluble film forming agents which do not comprise styrene, thickeners, coalescents, preservatives, fragrances, oils, waxes, surfactants, antioxidants, agents for combating free radicals, spreading agents, wetting agents, dispersing agents, antifoaming agents, neutralizing agents, stabilizing agents, active principles chosen from essential oils, UV screening agents, sunscreens, moisturizing agents, vitamins, proteins, ceramides, plant extracts, fibers, and the like, and their mixtures.

Suitable inorganic pigments can be white or colored, coated or uncoated. Mention may be made, for example, of inorganic pigments such as titanium dioxide, optionally surface treated, zirconium or cerium oxides and iron or chromium oxides, manganese violet, ultramarine blue, chromium hydrate and ferric blue. Suitable pearlescent pigments can be chosen from, for example, white pearlescent pigments, such as mica covered with titanium oxide or with bismuth oxychloride, colored pearlescent pigments, such as titanium oxide-coated mica with iron oxides, titanium oxide-coated mica with in particular ferric blue or chromium oxide, and pearlescent pigments based on bismuth oxychloride.

Specific examples of suitable water-soluble film forming agents not comprising styrene include, but are not limited to, proteins, such as proteins of plant origin, such as, for example, wheat or soya proteins; or proteins of animal origin, such as keratins, for example keratin hydrolysates and sulfonic keratins; cellulose polymers, such as, for example, hydroxyethylcellulose, hydroxypropylcellulose, methylcellulose or ethylhydroxyethylcellulose; vinyl polymers, such as, for example, polyvinylpyrrolidones, copolymers of methyl vinyl ether and of maleic anhydride, the copolymer of vinyl acetate and of crotonic acid, copolymers of vinylpyrrolidone and of vinyl acetate, copolymers of vinylpyrrolidone and of caprolactam, or polyvinyl alcohol; gums arabic, guar gum, xanthan derivatives or karaya gum; alginates and carrageenans; glycoaminoglycans, hyaluronic acid and its derivatives; shellac resin, gum sandarac, dammars, elemis or copals; muccopolysaccharides, such as chondroitin sulfates; and their mixtures.

A person skilled in the art will take care to select the optional additional additives and/or the amount thereof such that the advantageous properties of the composition according to the invention are not, or are not substantially, adversely affected by the envisaged addition.

These substances may be selected variously by the person skilled in the art in order to prepare a composition which has the desired properties, for example, consistency or texture.

These additives may be present in the composition in a proportion from 0% to 99% (such as from 0.01% to 90%) relative to the total weight of the composition and further such as from 0.1% to 50% (if present), including all ranges and subranges therebetween.

Needless to say, the composition of the invention should be cosmetically or dermatologically acceptable, i.e., it should contain a non-toxic physiologically acceptable. The composition may be in any galenic form normally employed in the cosmetic and dermatological fields which is suitable for topical administration onto nails.

According to preferred embodiments, a nail composition set comprising at least one nail composition and at least one topcoat is provided. The topcoat is optional in the present invention. However, if present, any topcoat suitable for application to nails as a topcoat can be used. That is, the topcoat employed in the nail composition sets of the present invention is not limited: as long as the topcoat is suitable for application to nails, it is suitable for the nail composition set of the present invention. Typically, topcoats provide shine and/or protection to color coats of nail composition set.

It should be understood that each coat or layer in the nail composition set, itself, can comprise one or more layers of each composition. Thus, the at least one nail composition can comprise one or more layers; and the at least one topcoat (if present) can comprise one or more topcoat layers. Preferably, each nail composition and topcoat contains three or fewer layers or compositions, more preferably two or fewer layers or compositions, and most preferably a single layer or composition.

According to preferred embodiments of the present invention, methods of making up or protecting nails comprising applying to the nails at least one nail composition of the present invention to nails in an amount sufficient to makeup or protect the nails are provided. According to preferred embodiments, at least one topcoat is further applied to the nails.

“Making up” as used herein means to provide decoration (for example, color) to the nail. “Protecting” as used herein means to inhibit damage to the nail (for example, chipping) by providing a protective layer on the nail.

In accordance with preferred embodiments of the preceding methods, at least one nail composition is applied topically to the nails of a person in need of (desirous) the desired making up or protection in an amount sufficient to achieve the desired result. The compositions may be applied to the desired area as needed.

According to preferred embodiments of the present invention, a kit for a nail composition set comprising at least one nail composition of the present invention are also provided. Preferably, the kit further comprises a topcoat composition.

According to preferred embodiments of the present invention, methods of minimizing staining of nails by a nail composition comprising water and at least one water-soluble organic coloring agent comprising adding at least one film forming agent comprising styrene to the nail composition in an amount sufficient to minimize staining of the nail are provided. Preferably, the amounts and ratios of ingredients added in these invention methods are the same amounts and ratios discussed above.

According to preferred embodiments of the present invention, methods of making a nail composition comprising combining ingredients comprising water, at least one water-soluble organic coloring agent, and at least one film forming agent comprising styrene to form a nail composition are provided. Preferably, the amounts and ratios of ingredients added in these invention methods are the same amounts and ratios discussed above.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective measurements. The following examples are intended to illustrate the invention without limiting the scope as a result. The percentages are given on a weight basis.

### Examples

### Example 1—Invention Compositions

Composition a—Sample Formulation

| | | Amount Range |
|---|---|---|
| INCI Name | Commercial Reference | (Active %) |
| Styrene/Acrylates Copolymer | Joncryl 77 (BASf) | 16-47% |
| Modified Styrene/Acrylates | Neocryl A-2091 (DSM) | 11-17% |
| Copolymer | | |
| Deionized Water | | 20-70% |
| Dipropylene Glycol | Lexfeel Shine (Inolex) | 1-7% |
| Dibenzoate | | |
| Tributyl citrate | Uniplex 83 (Unitex) | 1-5% |
| Phenoxyethanol | Neolone PH 100 (DOW) | 0.1-1% |
| Caprylyl glycol | Minacare Octiol | 0.1-0.7% |
| | (Minasolve) | |
| FD&C Blue 1 Aluminum lake | NNS FD&C Blue 1 lake | 1-3% |
| coated with methoxy | (Kobo) | |
| amodimethicone/ | | |
| silsesquioxane polymer | | |

Composition B—Pigment Paste

A pigment paste was prepared to disperse Sun Chemical Blue 1 Lake powder or Kobo Blue 1 Lake powder within the nail enamel formulation. Joncryl 77 (78 g) was added to a 100 g high speed mixer cup and stirred for 5 minutes using a propeller blade. Caprylyl glycol (1 g) and phenoxyethanol (1 g) were added slowly to the high speed mixer cup under vigorous stirring. The mixture was stirred for 5 minutes. Sun Chemical Blue 1 Lake powder or Kobo Blue 1 Lake powder (20 g) was added slowly in portions to the high speed mixer cup until the pigment was well suspended. The cup was then mixed using a Silversun for 30 minutes to disperse the pigment until there were no visible lumps.

Composition C—Invention Composition

The following composition was prepared:

| INCI Name | Commercial Reference | Amount Range |
|---|---|---|
| Styrene/Acrylates Copolymer | Joncryl 77 (BASf) | 59.8% |
| Modified Styrene/Acrylates | Neocryl A-2091 | 26.2% |
| Copolymer | (DSM) | |
| Dipropylene Glycol | Lexfeel Shine (Inolex) | 1% |
| Dibenzoate | | |
| Tributyl citrate | Uniplex 83 (Unitex) | 3% |
| Composition B - Pigment Paste | | 10% |

This composition was prepared by mixing the ingredients in a high speed mixer cup at 2500 RPM for 2 minutes.

Composition D—Comparative Composition

2 A staining water based nail enamel (Comparative 1) was formulated using the components shown in the table below. All components were mixed in a high speed mixer cup at 2500 RPM for 2 minutes.

| 1. INCI name | Weight % |
|---|---|
| Styrene/Acrylates/Ammonium methacrylate | 56.1 |
| Styrene/Acrylates Copolymer | 4.79 |
| Aqueous Polyurethane dispersion | 2.57 |
| propylene glycol butyl ether | 0.9 |
| dipropylene glycol dibenzoate | 3.15 |
| Tributyl Citrate | 0.45 |
| trimethyl hydroxypentyl isobutyrate | 0.45 |
| caprylyl glycol | 0.9 |
| phenoxyethanol | 0.9 |
| Modified Styrene Acrylate Copolymer | 6.3 |
| Ammonium acrylates copolymer | 13.5 |
| Composition B Pigment Paste | 10 |

### Example 2—Testing Protocols

Shine:

Shine properties were determined using a gloss meter. One layer of the composition to be tested was spread on a contrast card using an automatic spreader. The layer covered at least the white background of the card and was allowed to dry. Gloss was measured at 20° using a Byk Gardner gloss meter of reference microTRI-GLOSS. This measurement was repeated 3 times, and the average gloss (in gloss units (GUs)) is the average of the 3 measurements carried out.

Adhesion:

Adhesion properties of the films were determined using a Vitronail® substrate. Vitronails® from IMS Inc. (4 in.×3 in.) were cut and one layer of composition was applied and allowed to dry for 5 minutes. A second color coat layer was applied on top of the first and the nail enamel was allowed to dry overnight. The adhesion was assessed by determining how difficult it was to remove the product from the Vitronails® substrate using a spatula. The adhesion properties were assessed from 1 to 5, where 1 meant very difficult to remove and 5 very easy.

Hardness:

Film hardness was measured using the Byk scratch hardness test. The composition was coated on a glass plate using an automatic spreader. The composition was allowed to dry overnight. As is known in the art, pencils of varying degrees of hardness (6B pencil being the softest through 6H pencil being the hardest) were drawn over the surface of the film to determine which pencil caused indentation to determine film hardness. The resulting hardness was assessed from 1 to 5 with 1 being considered HB hardness and 5 being 2H hardness.

Staining:

Staining properties of the color coat compositions were assessed on Vitronail®. One color coat layer was applied and allowed to dry for 5 minutes. A second color coat layer was applied on top of the first and the nail enamel was allowed to dry overnight. The nail enamel was removed by soaking a cotton pad in acetone and allowing it to sit on the surface of the nail enamel and the amount of staining on Vitronail® was determined by visual interpretation. The staining properties were assessed from 1 to 5, where 1 meant excessive staining and 5 meant very little to no staining.

### Example 3—Testing

We mixed pigment pastes containing Sun Chemical Blue 1 Lake powder or Kobo Blue 1 Lake powder with over twenty different latex systems. We applied two coats of the blue 1 lake/latex formulas on to a Vitronail® substrate and evaluated whether the blue 1 dye would leach from each latex film and stain the substrate after 24 hours. Table 1 below shows the visual assessment of staining from blue 1/latex films on to Vitronail® substrate after the blue 1 lake/latex formulas were removed from nails (5=minimal staining; 1=severe staining).

TABLE 1

| | Commercial | Staining on Vitro |
|---|---|---|
| Latex (INCI name) | Name | Nail ® |
| Silane Modified Styrene Acrylic | Axilat DS 931 | 2 |
| Ester | | |
| Acrylates Copolymer | Balance CR | 3 |
| Acrylates Copolymer | Luviset One | 1 |
| Ammonium Acrylates | Vinysol 1086 WP | 1 |
| Copolymer | | |
| Styrene/Acrylates Copolymer | Neocryl XK 320 | 2 |
| Styrene/Acrylates Copolymer | Neocryl A-2091 | 5 |
| Acrylates Copolymer | Neocryl XK 102 | 3 |
| Styrene/Acrylates Copolymer | Joncryl 77 | 5 |
| Acrylates Copolymer | Neocryl A45 | 2 |
| Acrylates Copolymer | Demacryl C | 2 |
| Acrylates Copolymer | Epitex 66 | 1 |
| Styrene/Acrylates Copolymer | Syntran PC5620 | 2 |
| | CG | |
| Acrylates Copolymer | Acudyne Bold | 3 |
| Acrylates Copolymer | Aculyn 33 | 2 |
| Acrylates/VA copolymer | Vinysol 2140L | 2 |
| Acrylates/Hydroxyesters | Acudyne DHR | 2 |
| Acrylates Copolymer | | |

The above results indicated that two styrene acrylate copolymers (Joncryl 77 and Neocryl A-2091), when combined with a pigment paste containing Sun Chemical Blue 1 Lake paste or Kobo Blue 1 Lake showed the largest decrease in the amount of staining in vitro.

Then, thirty-two different water-based nail enamels containing different levels of these two styrene acrylate copolymers, along with different levels of plasticizer and coalescent, and along with a fixed amount of Blue 1 pigment paste were prepared. We assessed the formulas for adhesion, shine, consistency, film hardness and staining properties. The results are set forth in Table 2 below (5=good properties, 1=bad properties):

TABLE 2

| Formula | Shine | Adhesion | Staining | Hardness |
|---|---|---|---|---|
| 1 | 53.2 | 2 | 5 | 5 |
| 2 | 62 | 4 | 4 | 4 |
| 3 | 58.8 | 5 | 4 | 2 |
| 4 | 67.7 | 5 | 5 | 4 |
| 5 | 57.2 | 5 | 5 | 2 |
| 6 | 68.2 | 4 | 3 | 2 |
| 7 | 60.5 | 4 | 3 | 2 |
| 8 | 66.3 | 4 | 1 | 2 |
| 9 | 70 | 4 | 5 | 2 |
| 10 | 63.2 | 4 | 4 | 3 |
| 11 | 45.5 | 2 | 4 | 5 |
| 12 | 50.1 | 5 | 5 | 4 |
| 13 | 37.2 | 5 | 5 | 3 |
| 14 | 46.8 | 4 | 4 | 5 |
| 15 | 60.3 | 5 | 5 | 5 |
| 16 | 61.1 | 3 | 5 | 2 |
| 17 | 54.7 | 4 | 5 | 5 |
| 18 | 64.4 | 4 | 4 | 5 |
| 19 | 61.2 | 4 | 4 | 4 |
| 20 | 55.8 | 4 | 2 | 4 |
| 21 | 63.1 | 3 | 3 | 5 |
| 22 | 50.4 | 2 | 4 | 4 |
| 23 | 56.5 | 2 | 5 | 2 |
| 24 | 51.8 | 5 | 4 | 2 |
| 25 | 47.3 | 5 | 4 | 4 |
| 26 | 55.6 | 5 | 2 | 4 |
| 27 | 52.8 | 5 | 2 | 3 |
| 28 | 54.6 | 4 | 3 | 4 |
| 29 | 44 | 4 | 2 | 4 |
| 30 | 43.9 | 4 | 3 | 3 |
| 31 | X | 1 | 3 | X |
| 32 | 53.4 | 2 | 1 | 3 |
| 33 | 54.6 | 2 | 3 | 4 |
| 34 | 53.2 | 3 | 2 | 4 |
| 35 | 53.1 | 3 | 2 | 3 |
| 36 | 48.9 | 3 | 1 | 4 |
| 37 | 46.1 | 5 | 1 | 5 |
| 38 | 44 | 2 | 2 | 2 |
| 39 | 32.2 | 3 | 1 | 4 |

From the results set forth above, we generally determined that as we increased the amount of Joncryl 77, the amount of staining decreased. As we increased the amount of Neocryl A-2091, the amount of staining increased slightly. As we increased the amount of plasticizer, the amount of staining stayed the same and as we increased the amount of coalescent, the amount of staining increased dramatically.

We determined that Formula 15 (Composition C above) gave the best shine, adhesion, hardness and staining properties from the experiments.

What is claimed is:

1. A nail composition comprising water, at least one water-soluble organic coloring agent and at least one film forming agent comprising styrene.

2. The nail composition of claim 1, further comprising at least one plasticizer.

3. The nail composition of claim 1, further comprising at least one coalescent agent.

4. The nail composition of claim 2, further comprising at least one coalescent agent.

5. A nail composition set comprising (a) the nail composition set of claim 1 and (b) a topcoat composition.

6. The nail composition of claim 1, wherein water is present in an amount ranging from about 25% to about 70% by weight with respect to the total weight of the composition.

7. The nail composition of claim 1, wherein water-soluble organic coloring agent is present in an amount ranging from about 0.75% to about 7.5% by weight with respect to the total weight of the composition.

8. The nail composition of claim 1, wherein film forming agent comprising styrene is present in an amount ranging from about 10% to about 50% by weight with respect to the total weight of the composition.

9. The nail composition of claim 1, wherein the film forming agent comprising styrene and the water-soluble organic coloring agent are present in a weight ratio of about 50:1 to about 5:1.

10. The nail composition of claim 2, wherein the film forming agent comprising styrene and the water-soluble organic coloring agent are present in a weight ratio of about 50:1 to about 5:1.

11. The nail composition of claim 1, wherein the film forming agent comprising styrene contains 70% or more styrene by weight with respect to the total weight of the film forming agent.

12. The nail composition of claim 2, wherein plasticizer is present in an amount of less than 10% by weight with respect to the total weight of the composition.

13. The nail composition of claim 2, wherein plasticizer is present in an amount of less than 5% by weight with respect to the total weight of the composition.

14. The nail composition of claim 10, wherein plasticizer is present in an amount of less than 5% by weight with respect to the total weight of the composition.

15. The nail composition of claim 14, wherein the film forming agent comprising styrene contains 70% or more styrene by weight with respect to the total weight of the film forming agent.

16. The nail composition of claim 1, wherein the film forming agent comprising styrene and the coalescent agent are present in a weight ratio of about 50:1 to about 5:1.

17. A kit comprising (a) the nail composition set of claim 1 and (b) a topcoat composition.

18. A method of minimizing staining of nails by a nail composition comprising water and at least one water-soluble organic coloring agent comprising adding at least one film forming agent comprising styrene to the nail composition in an amount sufficient to minimize staining of the nail.
